(19) United States
(12) Patent Application Publication
(10) Pub. No.: US 2025/0303159 A1
(43) Pub. Date: Oct. 2, 2025

(54) DEVICE FOR PERFORMING TVNS TREATMENT

(71) Applicant: tVNS Technologies GmbH, Erlangen (DE)

(72) Inventor: Armin Bolz, Buckenhof (DE)

(21) Appl. No.: 19/234,933

(22) Filed: Jun. 11, 2025

Related U.S. Application Data
(63) Continuation-in-part of application No. 16/987,929, filed on Aug. 7, 2020, now Pat. No. 12,350,492.

(30) Foreign Application Priority Data
Aug. 9, 2019 (DE) 10 2019 121 583.8

(51) Int. Cl. A61N 1/36; A61N 1/04; G06F 3/16; G16H 10/60; G16H 20/40

(52) U.S. Cl. CPC A61N 1/36031; A61N 1/0456; G06F 3/165; G16H 10/60; G16H 20/40

(57) ABSTRACT

Systems are provided for a device. In one example, the device is configured for performing a transcutaneous vagus nerve stimulation (tVNS) treatment, the device including at least one electrode for generating a stimulation pulse, at least one acoustic stimulation device, an input device for inputting feedback data, an internal memory configured to store feedback, and a controller with instructions stored in memory thereof that when executed cause the controller to adjust one or more parameters of an acoustic stimulation provided by the at least one acoustic stimulation device.

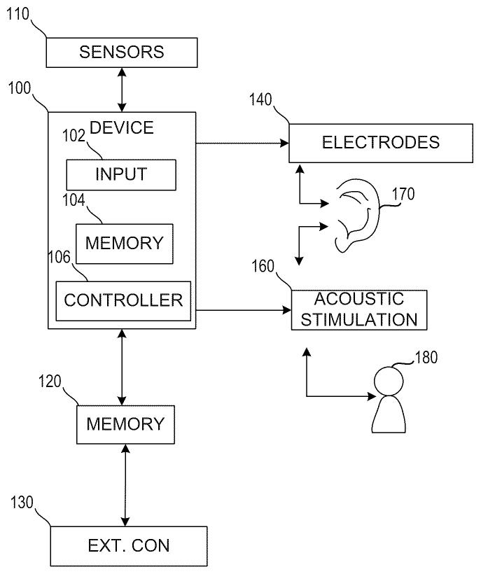

### CROSS REFERENCE TO RELATED APPLICATIONS

The present application is a continuation-in-part of U.S. Non-Provisional patent application Ser. No. 16/987,929, entitled “DEVICE FOR PERFORMING A TVNS TREATMENT”, and filed on Aug. 7, 2020. U.S. Non-Provisional patent application Ser. No. 16/987,929 claims priority to German Patent Application No. 10 2019 121 583.8 filed on Aug. 9, 2019. The entire contents of the above-listed applications are hereby incorporated by reference for all purposes.

### BACKGROUND OF THE INVENTION

The present invention relates to a device for performing a tVNS treatment in accordance with the description herein.

So-called transcutaneous vagus nerve stimulation (also called tVNS in the following) is known from the prior art as a treatment method that is based on a branch of the vagus nerve, namely the Ramus auricularis nervi vagi (RANV), being transcutaneously stimulated by electrical pulses. The method is used, for example, in the treatment of medically refractive epilepsy (MRE) and refractive depression.

The treatment is carried out with a device that generates electrical pulses that are delivered through the skin to said branch of the vagus nerve by an ear electrode that is worn like an earphone.

It is known from the prior art that the process of tVNS runs in accordance with a fixed stimulation protocol.

The device works through this stimulation protocol, that is stored in a memory, with fixed parameters after the switching on.

This procedure is inflexible to the extent that no optimization of the treatment is possible with respect to the wellbeing of the patient.

### SUMMARY OF THE INVENTION

It is therefore the underlying object of the invention to further develop a device of the initially named kind such that it enables an optimization of the treatment with respect to conventional devices.

This object is achieved by a device having the features described herein.

Provision is accordingly made that the device for performing a tVNS treatment is configured with at least one electrode for generating a stimulation pulse, wherein the device has at least one input device for inputting feedback data by the device user, wherein the device has a memory in which the feedback data are stored, and wherein the device has a control or regulation unit that is suitable to set one or more parameters of the stimulation pulse delivered by the electrode in dependence on the feedback data or to propose the parameters of the stimulation pulse delivered by the electrode for a selection by the device user, e.g. by display on a screen.

Instead of a few subjects as part of a defined study, in accordance with the invention, the effect of the device is optimized by feedback of the users while taking account of defined parameters.

The device knows the defined parameters of the pulse delivered by the electrode and learns by the feedback of the user or users whether these parameters have resulted in complaints such as headaches, etc. or whether an abatement or worsening has occurred or whether the wellbeing of the patient has improved or worsened.

Based on this feedback, the device can change the stimulation pulse on the next treatment or treatments such that ultimately a positive change occurs in the patient, i.e. an optimization is performed solely or inter alia on the basis of the feedback data.

The technical settings of the device such as the pulse duration, length, intensity, etc. are set in dependence on the feedback of the device user or users or are proposed for setting by the user so that he can decide himself whether he would like to follow the proposal.

Said optimization is possible for a large number of device users so that a very large amount of data can be obtained that allows a more targeted optimization of the parameter or parameters of the pulses delivered by the electrode.

The parameter or parameters of the stimulation pulse delivered by the electrode can represent its duration, strength, or frequency, or a combination of these parameters. Other possible parameters of the stimulation pulse can also be set in dependence on the feedback data or can be proposed.

It is conceivable that the feedback data stored in the memory are individual to the patient. This means that an optimization of the stimulation pulse individual to the patient is carried out.

It is also conceivable and covered by the invention that the feedback data stored in the memory are not individual to the patient, but rather relate to all the device users or to a majority of device users.

In this case, no setting of the stimulation pulse individual to the patient takes place, but the feedback data of a majority of users are rather detected and an optimization of the stimulation pulse is then carried out in dependence thereon for all device users or for a majority of device users.

The memory in which the feedback data are stored can be an integral component of the device or can be arranged as an external device component. This also applies in another respect to all the other components of the device.

In other words, the term “device” is not only to be understood as a compact device, but also as a system of components that do not necessarily have to be arranged at one and the same location. The memory can thus, for example, be formed as a data pool by a server that is spatially separate from that part of the device the user actuates.

It is conceivable that only a 2-point communication link from the input means to the memory is present.

It can be expanded by the inclusion of third parties such as physicians or psychologists and/or to other device users. These third parties are then able to provide additional advice how the treatment by the tVNS device can be further optimized.

The device can comprise means for delivering acoustic stimuli such as music or relaxation instructions to the device user.

Provision is preferably made here that the means have a communication link to the memory and the acoustic stimuli are stored in the memory, with the control or regulation unit being suitable to also set the one or more parameters of the stimulation pulse delivered by the electrode in dependence on the acoustic stimulus or stimuli or to propose these parameters for a selection by the device user.

The device is preferably portable and can in particular be held in the hand. It preferably has the size of a smartphone.

The kind of input device is as desired. A particularly simple design results if the input device is or comprises a touchscreen.

The device can have one or more sensors by means of which one or more patient parameters and/or external parameters can be detected, with the control or regulation unit being configured also to set one or more parameters of the stimulation pulse delivered by the electrode in dependence on the patient parameters and/or on the external parameters or to propose the parameters of the stimulation pulse delivered by the electrode for a selection by the device user.

In this case, these parameters such as the heart rate, weather conditions, etc. also enter into the parameter values of the stimulation pulse.

The electrode or electrodes are preferably an ear electrode.

Provision can be made in accordance with a method for the device for performing a tVNS treatment that feedback data that are stored in a memory are input by the device user by means of an input device and that one or more parameters of the stimulation pulse delivered by the electrode are set in in dependence on the feedback data or are proposed for a selection by the user.

### BRIEF DESCRIPTION OF THE DRAWING

The FIGURE shows an example of a tVNS device in communication with sensors, external memory, and stimulation devices.

### DESCRIPTION OF THE PREFERRED EMBODIMENTS

Preferred features of the method can be found in the description herein.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the invention will be explained in more detail with reference to an embodiment described in the following.

The perceived wellbeing of the user can be increased by the tVNS device in accordance with the invention or a perceptible improvement of a state experienced as negative can be achieved.

“Medical conditions” such as tinnitus, headache, depressive moods, obesity, etc. are meant by way of example as negatively experienced states.

Provision is made to achieve this perceptible improvement that the user provides feedback on his wellbeing during or after the treatment. These feedback data are stored and are used in a following treatment to optimize the parameter values of the delivered pulse such that the wellbeing of the patient is improved in the next treatment. This can be achieved, for example, in that the intensity or duration of the stimulation pulse is changed.

Further factors influencing the parameter values of the stimulation pulse can be external parameters such as the weather or the time of day; on the other hand, individual variables such as age, sex, but also preceding activities, perceived tiredness, perceived stress, etc.

Provision is preferably made to collect as many of these variables as possible in a first step. In addition, the technical settings have to be varied and correspondingly collected in a reproducible manner.

All these collected data and the feedback data form a large data pool. This data pool has to be analyzed—in particular for positive and negative links between the variables with respect to their effects on the perceived wellbeing or the perceived change of a negative state.

As a result, recommended actions are formed for the individual user, optionally also for whole user groups. These recommended actions are continuously optimized on the basis of the data present.

Studies of the previously customary kind can be dispensed with using this procedure. The outlined data pool-oriented procedure is rather superior due to the individual tailoring of the results and the continuous optimization of the results.

Any uncertainty that may be present in the collection of the data on the user's own perception and on the further factors is more than compensated by the amount and constant updating of the data.

Conceivable applications of the method or of the tVNS device in accordance with the invention are e.g.
tinnitus
quitting smoking
headache/light migraines
falls in concentration
being overweight
mild dementia
depressive moods
stress reduction
eating disorders
high blood pressure.

All these parameters or also individual ones of them can be perceived by the patient and reported back to the device as feedback.

The control or regulation unit can generally be configured to carry out an optimization directly for a specific parameter such as headaches, or, in the ideal case, to carry out the optimization such that the symptoms of the patient improve overall.

Turning now to the FIGURE, it shows an embodiment of a device 100. The device 100 may include multiple components and/or modules that are arranged at a single location or multiple locations. The device 100 may include an input device 102, memory 104, and a controller 106.

The input device 102 may be configured to receive feedback data directly from a user and/or a user device. Additionally, or alternatively, the input device 102 may be a touchscreen or other display configured to receive inputs. In some examples, the feedback may be provided in response to survey questions or prompts. Such feedback may be captured through structured formats, including standardized numerical surveys, multiple-choice selections, rating systems, free-form textual responses, or the like. The feedback may be stored in memory 104. The memory 104 may be internal to the device 100. In one example, the memory 104 is non-transitory memory. The device 100 may include a controller 106. The controller 106 may be configured, via instructions stored in memory 120, to change one or more parameters of a stimulation pulse or an acoustic stimulation during a next treatment and/or next treatments. The parameters of the stimulation pulse may include one or more of a pulse duration, length, intensity, and the like. The parameters of the acoustic stimulation may include one or more of a volume (e.g., bass volume, treble volume, overall volume), a frequency, a music type, a noise type, an order, and a location at which the acoustic stimulus is provided.

The device 100 may include a plurality of sensors 110. The plurality of sensors 110 may be integrally arranged in the device 100 and/or communicatively coupled thereto. The plurality of sensors 110 may include a wired or wireless connection to the device 100. A wired connection may include a cable or other element configured to transfer signals between the device 100 and the plurality of sensors 110. Wireless communication may include one or more of Wi-Fi, Bluetooth, radio broadcasting, radar communication, and radio frequency identification. The plurality of sensors 110 may be configured to monitor user data, such as biometric data, including but not limited to one or more of a breathing rate, relaxation exercises (stress, stimulus load, sleep duration, sleep quality, and the like), physical exercises (step count, distance moved, activity completed, and the like), electroencephalogram (EEG), and electrocardiography (ECG).

The memory 120 may be configured as external memory. In one example, the memory 120 is configured as an external cloud or server in wired or wireless communication with the device 100. The memory 120 may be in wired or wireless communication with an external controller 130. The external controller 130 may be configured to execute an application. The application may be configured to perform data analysis, wherein data for the data analysis is based on metrics gathered by the plurality of sensors 110 and feedback received by the input device 102. The data is stored in memory 104 of the device 100 and provided to the memory 120 so that the application may analyze the data.

The application may be further configured to generate treatment specific profiles for a patient's medical condition. For example, a treatment profile related to depression may be different than a treatment profile related to epilepsy, tinnitus, migraines, obesity, and/or chronic pain. The application may, for example, be configured to generate and model customized audio-data for the patient's medical condition. The customized audio-data may be associated with the treatment specific profile.

The application may be further configured to generate customized music for the patient. The customized music may be generated via artificial intelligence (AI). In one example, the AI may be executed via a machine leaning model, such as a feedforward neural network, a convolutional neural network, a recurrent neural network, or a long short-term memory network. The customized music may include notch music therapy, sound therapy, neuromodulatory music, binaural beats, pink noise, white noise, brown noise, pre-existing songs, and AI generated songs. Generating customized music for the patient may involve taking into account the patient's known musical preferences.

In some examples, based on the feedback from the patient, the music selection for a medical condition of a first patient may be used for a second patient with a same medical condition. The music selection may be used as a starting treatment for the second patient, wherein the treatment is modified based on feedback from the second patient.

The device 100 may be coupled to each of an electrode 140 and an acoustic stimulation device 150. The electrode 140 may be configured to provide electrical stimulation of the vagus nerve. The acoustic stimulation device 150 may be configured to provide an acoustic stimulation. In some examples, the acoustic stimulation may be sounds, harmonics, music, and the like. The electrode 140 and the acoustic stimulation device 150 may be coupled to an ear 170. The ear 170 may represent a left ear or a right ear of the patient. In some examples, the electrode 140 and the acoustic stimulation device 150 may be coupled to both ears of the patient. Additionally, or alternatively, the electrode 140 and the acoustic stimulation device 150 may be coupled to a body 180 or other area of the patient. The acoustic stimulation device 150 may be headphones, musical instruments configured to contact the patient's body, musical instruments configured to provide audio stimulation without contacting the patient's body, singing bowls, a second electrode, and the like.

The device 100 may be one of a plurality of devices, wherein a first patient is associated with the device 100 and a treatment profile for the patient is saved in the memory 104. A plurality of patients may be associated with others of the plurality of devices. Treatment profiles may be stored in memory of the others of the plurality of devices. In some examples, patients with overlapping or similar conditions may include similar or identical treatment profiles. The application may be configured to analyze variables of a data pool for whether there are positive and negative links between the variables with respect to their effects on the perceived wellbeing and generate an analyzed result, wherein the variables include weather, time of day, age, sex, preceding activity, perceived tiredness, and perceived stress, and wherein the one or more parameters of the stimulation pulse and the acoustic stimulus are adjusted based on the analyzed variables. Thus, as an example, if the first patient experiences a positive outcome based on a treatment modification, then the treatment modification may be provided to other patients with similar or identical conditions. Alternatively, if the first patient experiences a negative outcome based on the treatment modification, then the treatment modification may not be provided or may be removed from treatment plans of other patients with similar or identical conditions.

In one example, the external controller 130 may be configured to operate as a central controller, wherein the controller 106 of the device 100 may be omitted. By omitting the controller 106, a manufacturing cost of the device 100 may be reduced. The external controller 130 may signal to sensors of the separate devices to execute stimuli

Note that the example control and estimation routines included herein can be used with various system configurations. The control methods and routines disclosed herein may be stored as executable instructions in non-transitory memory and may be carried out by the control system including the controller in combination with the various sensors, actuators, and other hardware. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein, but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of the computer readable storage medium in the control system, where the described actions are carried out by executing the instructions in a system including the various hardware components in combination with the electronic controller.

It will be appreciated that the configurations and routines disclosed herein are exemplary in nature, and that these specific embodiments are not to be considered in a limiting sense, because numerous variations are possible. The subject matter of the present disclosure includes all novel and non-obvious combinations and sub-combinations of the various systems and configurations, and other features, functions, and/or properties disclosed herein.

As used herein, the term “approximately” is construed to mean plus or minus five percent of the range unless otherwise specified.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A device for performing a transcutaneous vagus nerve stimulation (tVNS) treatment, the device comprising:
at least one electrode for generating a stimulation pulse;
at least one acoustic stimulation device;
an input device for inputting feedback data;
an internal memory configured to store feedback; and
a controller with instructions stored in memory thereof that when executed cause the controller to:
adjust one or more parameters of an acoustic stimulation provided by the at least one acoustic stimulation device.

2. The device of claim 1, wherein the acoustic stimulation device and the memory are communicatively coupled.

3. The device of claim 1, wherein the instructions further cause the controller to adjust one or more parameters of the stimulation pulse delivered by the electrode in dependence on the feedback data.

4. The device of claim 1, wherein the acoustic stimulation device is one of a pair of headphones, a musical instrument configured to contact a user's body, a musical instrument configured to operate without contacting the user's body, or singing bowls.

5. The device of claim 1, wherein the acoustic stimulation device is configured to provide an acoustic stimulation of a user's ear.

6. The device of claim 1, wherein the acoustic stimulation device is configured to provide acoustic stimulation to a user's body.

7. The device of claim 1, wherein the instructions further cause the controller to generate a specific treatment profile based on the feedback data.

8. A system for performing a transcutaneous vagus nerve stimulation (tVNS) treatment, the system comprising:
a device comprising a memory and a feedback module, wherein the memory is configured to store feedback input by the feedback module;
at least one electrode configured to generate a stimulation pulse, the at least one electrode communicatively coupled to the device;
at least one acoustic stimulation device configured to generate an acoustic stimulus, the at least one acoustic stimulation device communicatively coupled to the device; and
a controller with instructions stored in memory thereof that when executed cause the controller to:
adjust one or more parameters of the stimulation pulse delivered by the electrode in response to the feedback data; and
adjust one or more parameters of the acoustic stimulus provided by the at least one acoustic stimulation device in response to the feedback data.

9. The system of claim 8, wherein the instructions further cause the controller to generate customized music as the acoustic stimulus.

10. The system of claim 8, wherein the instructions further cause the controller to use a music selection for a condition of a first patient for a second patient with the condition.

11. The system of claim 8, wherein the at least one acoustic stimulation device is coupled to a left ear, a right ear, or both ears of a patient.

12. The system of claim 8, wherein the at least one acoustic stimulation device is configured to provide the acoustic stimulation to a body of a patient other than via an ear.

13. The system of claim 8, wherein the device is in communication with one or more sensors, wherein the one or more sensors is/are configured to monitor patient data.

14. The system of claim 8, wherein one or more parameters of the acoustic stimulus that is/are adjusted comprises a volume, a frequency, a music type, a noise type, an order, and a location at which the acoustic stimulus is provided.

15. The system of claim 8, wherein the memory stores feedback related to individual patient associated with the device and from other patients associated with other devices, and wherein the instructions further cause the controller to adjust one or more parameters of the stimulation pulse and the acoustic stimulus based on an abatement or worsening of the individual patient's or other patient's symptoms.

16. A system for performing a transcutaneous vagus nerve stimulation (tVNS) treatment, the system comprising:
a device comprising a memory and an input module, wherein the memory is configured to store feedback input via the input module;
at least one electrode configured to generate a stimulation pulse, the at least one electrode communicatively coupled to the device;
at least one acoustic stimulation device configured to generate an acoustic stimulus, the at least one acoustic stimulation device communicatively coupled to the device; and
a controller with instructions stored in memory thereof that when executed cause the controller to:
generate a data pool based on feedback provided to the device via the input module and to other devices identical to the device via other input modules;
adjust one or more parameters of the stimulation pulse delivered by the electrode in response to the feedback data or to propose the parameters of the stimulation pulse delivered by the electrode for selection by the device user; and
adjust one or more parameters of the acoustic stimulus provided by the at least one acoustic stimulation device.

17. The system of claim 16, wherein the instructions further cause the controller to analyze variables of the data pool for whether there are positive and negative links between the variables with respect to their effects on the perceived wellbeing and generate an analyzed result, wherein the variables include weather, time of day, age, sex, preceding activity, perceived tiredness, and perceived stress, and wherein the one or more parameters of the stimulation pulse and the acoustic stimulus are adjusted based on the analyzed variables.

18. The system of claim 16, wherein the at least one acoustic stimulation device includes a second electrode separate from the electrode.

19. The system of claim 16, further comprising an external controller configured to execute an application via instructions stored in an external memory, wherein the application, the control unit, and the external memory are arranged outside of the device, wherein the control unit is configured to communicate with the controller of the device and to controllers of other devices.

20. The system of claim 19, wherein the application is configured to generate a treatment plan for a patient associated with the device and use the treatment plan for other patients with one or more conditions similar to the patient.
